(19) United States
(12) Patent Application Publication
(10) Pub. No.: US 2026/0198827 A1
(43) Pub. Date: Jul. 16, 2026

(54) METHOD AND APPARATUS FOR IN VIVO ELECTRIC FIELD IMAGING OF NEURONS AT LOW MAGNETIC FIELD STRENGTHS

(71) Applicant: Weinberg Medical Holdings, Inc., North Bethesda, MD (US)

(72) Inventor: Irving N. WEINBERG, Chevy Chase, MD (US)

(21) Appl. No.: 19/448,789

(22) Filed: Jan. 14, 2026

Related U.S. Application Data
(60) Provisional application No. 63/744,969, filed on Jan. 14, 2025.

(51) Int. Cl. A61B 5/246; A61B 5/00; G01R 33/50

(52) U.S. Cl. CPC A61B 5/246; A61B 5/0051; G01R 33/50

(57) ABSTRACT

An apparatus and method are provided for collecting data used to create images of electrical fields in neuronal tissues. One or more modules of the apparatus have at least one electropermanent magnet, at least one radiofrequency antenna, and a means of neuromodulation; . A control system controls each of the one or more modules by using the nuclear quadrupole resonance phenomenon of the neuronal tissues to modulate and to image electrical field activity in the neuronal tissues

### CROSS-REFERENCE AND PRIORITY CLAIM

This application claims priority to United States Provisional Patent Application Ser. No. 63/744,969, entitled “METHOD AND APPARATUS FOR IN VIVO ELECTRIC FIELD IMAGING OF NEURONS AT AMBIENT LOW MAGNETIC FIELD STRENGTHS” filed Jan. 14, 2025, the entirety of which is incorporated by reference.

### BACKGROUND:

Nuclear quadrupole resonance (NQR) is typically used for examining the chemical state of elements with spin higher than ½. The NQR signal exists because of an interaction between the nuclear spin and the strong electric field gradient (EFG) from local electron clouds around the nucleus. NQR is generally not affected by the external electrical or magnetic fields that can be produced with magnetic resonance imaging (MRI) systems, because those fields are not as strong as the EFG field within the atom (which is in the range of kilovolts per meter). NQR is often detected by transmitting a radiofrequency (RF) pulse sequence into a sample, and then receiving RF signals from the sample. NQR signal is generally not detectable in liquids but is may be detected from anisotropic ordered organic structures such as folded DNA or eye lenses, as discussed by J. Schultz, L. Nordenskiöld, and A. Rupprecht, “A study of the quadrupolar NMR splittings of⁷ Li⁺,²³ Na⁺, and¹³³ Cs⁺counterions in macroscopically oriented DNA fibers,” Biopolymers, vol. 32, no. 12, pp. 1631-1642, Dec. 1992, and by A. Stevens, P. Paschalis, and T. Schleich, “Sodium-23 and potassium-39 nuclear magnetic resonance relaxation in eye lens. Examples of quadrupole ion magnetic relaxation in a crowded protein environment,” Biophys. J., vol. 61, no. 5, pp. 1061-1075, May 1992.

An indirect way of detecting NQR is to start with a quasi-static magnetic field (e.g., 100 milliTeslas) to polarize both protons and the nucleus of interest (e.g., Na-23). The static field may be turned off (in one or more sections of) the sample, and RF energy at the NQR resonance of the nucleus of interest may be applied to (one or more sections of) the sample, thereby decreasing the polarization of the nucleus of interest. The sample is then returned to a relatively high static field and the protons'degree of magnetization is affected by the residual magnetization of the nucleus of interest, thereby creating an image of the nucleus of interest as detailed in Lee, Y and Butler, L.G., “Field-Cycling 14N NQR Imaging with Spatial and Frequency Resolution,” J Magn. Reson., vol. 112, no. 1, pp. 92-95, 1995. Another method is to collect relaxation maps of protons at various frequencies, which (due to transfer of magnetization from NQR nuclei to protons or vice versa) exhibit anomalous behavior at NQR resonant frequencies as detailed in V. Mallikourti, P. J. Ross, O. Maier, K. Hanna, E. Husain, G. R. Davies, D. J. Lurie, G. Lip, H. Lahrech, Y. Masannat, and L. M. Broche, “Field cycling imaging to characterise breast cancer at low and ultra-low magnetic fields below 0.2 T,” Commun. Med., vol. 4, no. 1, p. 221, Oct. 2024

### SUMMARY

Disclosed embodiments relate to an apparatus and method for using NQR signals to detect strong electrical fields in neuronal tissue, for example, as found in the brain.

An apparatus for collecting data used to create images of electrical fields in neuronal tissues may comprise one or more modules having at least one electropermanent magnet, at least one radiofrequency antenna, and a means of neuromodulation; and a control system to control each of the one or more modules by using the nuclear quadrupole resonance phenomenon of the neuronal tissues to modulate and to image electrical field activity in the neuronal tissues.

The means of neuromodulation may be a current-carrying structure. The means of neuromodulation may be generator of ultrasound.

The control system may control a radiofrequency transmitted into neuronal tissues to primarily affect protons, and an imaging contrast is obtained through excitation of nuclei with spin greater than one-half.

The control system may control the at least one electropermanent magnet so that the magnetic field applied by the at least one electropermanent magnet creates a spinning magic angle effect that influences magnetic polarization transfer.

A method for collecting data used to create images of electrical fields in neuronal tissues may comprise providing one or more modules having at least one electropermanent magnet, at least one radiofrequency antenna, and a means of neuromodulation; and modulating neuronal tissues to increase or decrease electrical fields within the neuronal tissues, wherein an electrical field within the neuronal tissues affects the relaxation of nuclei of the neuronal tissues via nuclear quadrupole resonance and is measured with radiofrequency antenna.

The method may further comprise controlling the at least one electropermanent magnet so that the magnetic field applied by the at least one electropermanent magnet creates a spinning magic angle effect that influences magnetic polarization transfer.

The radiofrequency transmitted into neuronal tissues may primarily affect protons, and imaging contrast may be obtained through excitation of the protons and subsequent transfer of magnetization from nuclei with spin greater than one-half to the protons. The radiofrequency transmitted into neuronal tissues may primarily affect protons, and an imaging contrast may be obtained through excitation of nuclei with spin greater than one-half.

The modulating may be applied before collecting data that may be used to create images of the electrical field of neurons of the neuronal tissues. The modulating may be applied after collecting data that may be used to create images of the electrical field of the neurons of the neuronal tissues.

The images may yield functional magnetic resonance images of the neuronal tissues of the brain at low magnetic fields.

In some embodiments, a method for collecting data used to create images may comprise controlling at least two electropermanent magnets so that the magnetic field applied by the at least two electropermanent magnets creates a spinning magic angle effect without the need to physically spin the object of interest.

### BRIEF DESCRIPTION THE FIGURES

FIG. 1 is an embodiment of the apparatus positioned around neuronal tissue in a subject;

FIG. 2 shows a flowchart of a method for collecting data used to create images of electrical fields in neuronal tissues according to the disclosed embodiments.

### DETAILED DESCRIPTION

As detailed above, an NQR signal exists because of an interaction between a nuclear spin and a strong electric field gradient (kV/m) from local electron clouds. Disclosed embodiments exploit the fact that although the voltages within neuronal tissues during neuronal excitation (e.g., action potentials) are about a tenth of a volt, the distances upon which these voltages occur is very small (i.e., microns or less). As a result, the electrical field across the membranes of neuronal tissues during such excitation can be very high (e.g., 10 million volts per meter), so that the interaction between the nucleus and the local electrical field gradient may be affected by neuronal excitation. By modulating the neuronal tissues (e.g., with transcranial magnetic stimulation, or “TMS”), it is possible to collect NQR data from many neurons at once, thereby increasing the signal-to-noise ratio of the NQR data collected.

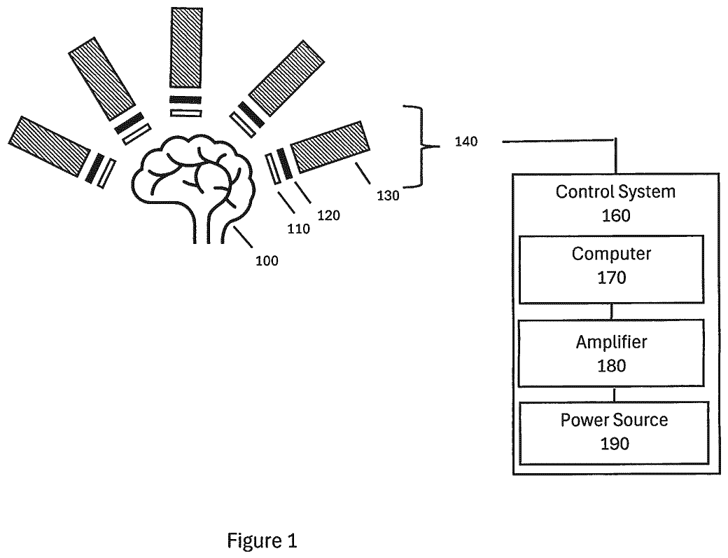

As illustrated in FIG. 1, the apparatus may have one or more modules 140 having one or more radiofrequency antennas 110. Neuronal tissue 100 of a subject, such as a human or animal, may be positioned near (e.g., less than 1 meter away) one or more RF antennas 110, neuromodulation means 120 such as a coil, and electropermanent magnets 130. One or more of RF antennas 110, neuromodulation means 120, and electropermanent magnets 130 may be incorporated into a module 140. Multiple modules 140 surround the neuronal tissue. A control device 160 incorporating one or more of computers 170, amplifiers 180, power sources 190, wires, and other components or systems needed may be coupled wired or wirelessly to the one or more modules 140 to control and power the modules 140.

The apparatus may have one or more electropermanent magnets, each of which can contribute to a magnetic field manifest over a field of view that includes the neuronal tissue. The superposition of these magnetic fields can create a quasi-static magnetic field (or spinning magnetic field) whose proton frequencies are near the expected NQR resonance frequencies for nuclei of interest (for example, Na-23 or N-14) in the neural tissue. In such cases, spin magnetization can transfer from protons to the nuclei of interest thereby providing imaging contrast. In some embodiments, the apparatus includes means for applying neuromodulation to neurons, for example with current-carrying structures (e.g., TMS coils). For the purposes of this disclosure, the term “current-carrying structures” includes coils of wires, metallic traces, or other assemblies of materials that can carry electrical currents. In some embodiments, the apparatus can null the magnetic field within a section of the sample (e.g., in a brain tract) to selectively collect NQR information from that tract, as taught by Lee and Butler as described above. A series of NQR images can assess the magnitude of the electric field in the neuronal tissue.

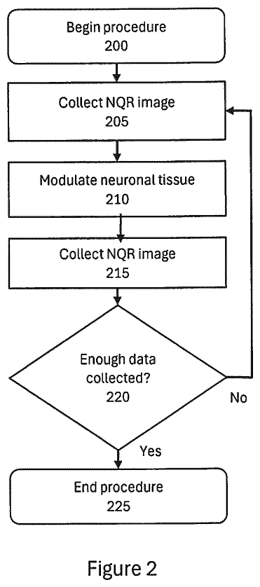

FIG. 2 describes a method of creating magnetic rotations near the frequencies of magnetic resonances at or near the expected NQR resonant within the apparatus shown in FIG. 1. Disclosed embodiments use the same apparatus to stimulate neurons to fire and to collect magnetic resonance images from the sample. A procedure, for example a theranostic study, may be initiated 200. An NQR image is obtained of one or more sections of neuronal tissue 205. The neuronal tissue is modulated (e.g., stimulated with TMS) to increase or decrease the electrical fields within the one or more sections of neuronal tissue 210. An NQR image is again obtained 215. An assessment is made by a computer or human operator as to whether enough data have been collected 220 to end the procedure 225.

Magic-angle spinning has been previously performed by spinning a sample (e.g. tissue) in a fixed magnetic field. In the disclosed embodiments, the use of electropermanent magnets allows the quasi-static magnetic field to spin around the fixed sample. In other words, magic-angle spinning in disclosed embodiments is performed by actuating the electropermanent magnets at different times so that the quasi-static magnetic field is spinning, and therefore the object of interest does not need to be spun. In some embodiments, a method of controlling at least two electropermanent magnets for imaging is provided so that the magnetic field applied by the at least two electropermanent magnets creates a spinning magic angle effect without the need to physically spin the object of interest (i.e. the object of interest remains stationary). In some embodiments the method may be performed as part of an MRI imaging sequence or as part of an NQR-sensitive MRI imaging sequence.

In some embodiments, the method may include the application of neuromodulation to neurons after the NQR-sensitive imaging sequences. In some embodiments, the method may include collecting an MRI of the one or more sections of neuronal tissue before neuromodulation, so as to collect information about the resting state of the neuronal tissues that may assist in planning the neuromodulation procedure. In some embodiments, the method includes collecting an MRI of the neuronal tissues before and after neuromodulation, which may be used to assess the dose of the neuromodulation delivered. In some embodiments, the method includes measuring the T1 or other relaxation time of protons above and below the expected NQR resonant frequencies of sodium-23 or another nucleus with spin above ½.

For the purposes of this disclosure, the terms quasi-static magnetic field or field-cycling are used to describe the application of magnetic fields that last long enough to affect polarization of nuclei but may change in magnitude. The term electropermanent magnet means a combination of magnetizable materials and a coil (or other carrier of electrical current) so that current through the coil will alter the magnetization of the magnetizable materials, and the magnetization will remain until another current is applied to the coil. In some embodiments, the magnetic field created by the electropermanent magnet may be supplemented by (or subtracted by) a permanent magnet.

In some embodiments, the means of neuromodulation may be a coil for transcranial magnetic or electrical stimulation, or a generator of focused ultrasound.

For the purposes of this disclosure, the term “low magnetic field” refers to a quasi-static magnetic field of less than one Tesla. For the purposes of this disclosure, the term “radiofrequency emitter” refers to a transmitter of radiofrequency energy. It is understood that an alternative to radiofrequency emission is to change the direction of the quasi-static field, as may be done with electropermanent magnets.

Those skilled in the art will recognize, upon consideration of the above teachings, that the above exemplary embodiments and the controller may be based upon use of one or more programmed processors programmed with a suitable computer program. However, the disclosed embodiments could be implemented using hardware component equivalents such as special purpose hardware and/or dedicated processors. Similarly, general purpose computers, microprocessor-based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out the above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

1. An apparatus for collecting data used to create images of electrical fields in neuronal tissues comprising:
one or more modules having at least one electropermanent magnet, at least one radiofrequency antenna, and a means of neuromodulation; and
a control system to control each of the one or more modules by using the nuclear quadrupole resonance phenomenon of the neuronal tissues to modulate and to image electrical field activity in the neuronal tissues.

2. The apparatus as in claim 1, wherein the means of neuromodulation is a current-carrying structure.

3. The apparatus as in claim 1, wherein the means of neuromodulation is a generator of ultrasound.

4. The apparatus of claim 1, wherein the control system controls a radiofrequency transmitted into neuronal tissues to primarily affect protons, and an imaging contrast is obtained through excitation of nuclei with spin greater than one-half.

5. The apparatus of claim 1, wherein the control system controls the at least one electropermanent magnet so that the magnetic field applied by the at least one electropermanent magnet creates a spinning magic angle effect.

6. A method for collecting data used to create images of electrical fields in neuronal tissues, the method comprising:
providing one or more modules having at least one electropermanent magnet, at least one radiofrequency antenna, and a means of neuromodulation;
modulating neuronal tissues to increase or decrease electrical fields within the neuronal tissues, wherein an electrical field within the neuronal tissues affects the relaxation of nuclei of the neuronal tissues via nuclear quadrupole resonance and is measured with radiofrequency antenna.

7. The method of claim 6, wherein the radiofrequency transmitted into neuronal tissues primarily affects protons, and imaging contrast is obtained through excitation of the protons and subsequent transfer of magnetization from nuclei with spin greater than one-half to the protons.

8. The method of claim 6, wherein a radiofrequency transmitted into neuronal tissues primarily affects protons, and an imaging contrast is obtained through excitation of nuclei with spin greater than one-half.

9. The method of claim 6, wherein the modulating is applied before collecting data that may be used to create images of the electrical field of neurons of the neuronal tissues.

10. The method of claim 9, wherein the modulating is applied after collecting data that may be used to create images of the electrical field of the neurons of the neuronal tissues.

11. The method of claim 9, wherein the images yield functional magnetic resonance images of the neuronal tissues of the brain at low magnetic fields.

12. A method for collecting data used to create images comprising:
controlling at least two electropermanent magnets so that the magnetic field applied by the at least two electropermanent magnets creates a spinning magic angle effect without the need to physically spin the object of interest.
